(19) United States
(12) Patent Application Publication
(10) Pub. No.: US 2007/0213390 A1
(43) Pub. Date: Sep. 13, 2007

(54) HMGCoA reductase inhibitor-angiotensin converting enzyme inhibitor compounds

(75) Inventors: Tadeusz Cynkowski, Brookline, MA (US); Grazyna Cynkowska, Brookline, MA (US); Thomas J. Smith, Altadena, CA (US)

Correspondence Address: FISH & NEAVE IP GROUP;ROPES & GRAY LLP, ONE INTERNATIONAL PLACE, BOSTON, MA, 02110-2624, US

(73) Assignee: pSivida Inc., Watertown, MA (US)

(21) Appl. No.: 11/724,149

(22) Filed: Mar. 14, 2007

Related U.S. Application Data
(63) Continuation of application No. 11/495,173, filed on Jul. 28, 2006.
(60) Provisional application No. 60/704,263, filed on Aug. 1, 2005.

(51) Int. Cl. A61K 31/401; A61K 31/366; A61K 31/22; A61K 31/4184

(52) U.S. Cl. 514/423; 514/460; 514/548; 514/394; 514/381

(57) ABSTRACT

A compound that contains at least two independently active pharmacological moieties, either covalently conjoined through a physiologically labile linker or ionically associated. One pharmacological moiety is an HMGCoA reductase inhibitor (such as a statin). Another pharmacological moiety is an angiotensin converting enzyme inhibitor.

### CROSS REFERENCE TO RELATED APPLICATIONS

This application claims priority under 35 U.S.C. § 119 based on U.S. provisional Application Ser. No. 60/704,263 filed Aug. 1, 2005, the disclosure of which is incorporated herein by reference.

### BACKGROUND OF THE INVENTION

Cardiovascular diseases, which include coronary heart disease and stroke, are the leading causes of death in the United States. The major risk factors of cardiovascular diseases are high blood cholesterol, high blood pressure (hypertension), and smoking and dietary factors. Stamler J., Established Major Coronary Risk Factors. In: Coronary Heart Disease Epidemiology. From Aetiology To Public Health, Marmot M & Elliott P, eds., 35-66 (Oxford University Press, New York, 1992). Elevated blood cholesterol is a major risk factor for coronary heart disease, and hypertension is the major risk factor for stroke. Hypertension can also increase the risk of myocardial infarct. Many clinical trials have demonstrated the efficacy of antihypertensive and lipid-lowering drugs for treating cardiovascular diseases. National Institutes of Health. The Sixth Report Of The Joint National Committee On Prevention, Detection, Evaluation, And Treatment Of High Blood Pressure. NIH publication no. 98-4080 (Rockville, Md.: US Department of Health and Human Services, National Institutes of Health, National Heart, Lung, and Blood Institute, November 1997); National Cholesterol Education Program. Second Report Of The Expert Panel On Detection, Evaluation And Treatment Of High Blood Cholesterol In Adults. NIH publication no. 93-3095 (Rockville, Md.: US Department of Health and Human Services, National Institutes of Health, 1993).

However, some important indicators of risk for cardiovascular disease have not improved recently, have leveled off, or are reversing. For example, approximately 70% of persons with hypertension do not have the condition controlled at levels below 140/90 mm Hg, and death rates for stroke have not declined in recent years. National Heart, Lung and Blood Institute. Morbidity & Mortality: 1998 Chartbook On Cardiovascular, Lung, And Blood Diseases. (Rockville, Md.: US Department of Health and Human Services, National Institutes of Health, 1998; Higgins M & Thom T, Int. J. Epidemiol. 1989; 18:S58-S66). Heart failure has emerged as a health concern for older adults (CDC, MMWR 47:633-7 (1998)), and adults who survive a myocardial infarction or other hypertension-related diseases remain at increased risk for heart failure.

Medications can only be effective if patients comply with their therapeutic regimen. The problem of patient noncompliance with medication use remains one of the most significant issues facing our health care system. The negative impact of noncompliance on patient outcomes has been documented for patients with hypertension. Morse, G. D. et al., Am. J. Hosp. Pharm. 43:905-909 (1986). Conversely, there is good evidence that patients who are more compliant in taking antihypertensive medications are more likely to achieve blood pressure control. Caro, J. J. & Speckman, J. L., J. Hypertension. 16:S31-S34 (1998).

The availability of several different drug targets for controlling hypertension has offered the potential of multiple-drug regimens. Polypharmacy is difficult to avoid, because using one drug can control blood pressure in only about 50% of patients. However, such multiple-drug regimens reduce patient compliance. Oparil, S. & Calhoun, D. A, American Family Physician, 1007 (Mar. 1, 1998). As a partial solution to this problem, fixed-dose combination therapy is designed to improve patient compliance by decreasing the number of pills that must be taken and reducing the dose-dependent adverse effects of individual components. Sica, D A., Drugs 48:16-24 (1994). To be combined in a single-dose form, however, U.S. law requires that each component in the combination must contribute to therapeutic effect and that the dosage of each component must be such that the combination is safe and effective in a major proportion of the target population (i.e., patients whose hypertension is not easily controlled with a single drug). 21 C.F.R. § 300.50 (the “fixed combination” policy). See also, 21 C.F.R. § 330.10(a)(4)(iv).

Accordingly, there is a continuing need in the medical arts for pharmaceutical compounds that deliver two or more drugs that are effective for treating cardiovascular disease at a single time in a single dose, to enhance patient compliance.

### SUMMARY OF THE INVENTION

The invention provides a compound comprising a first pharmacological moiety connected to, at least, a second pharmacological moiety through a physiologically labile linker, or a salt thereof, wherein both pharmacological moieties, when active or when activated, act to reduce cardiovascular disease. The first pharmacological moiety is an HMGCoA inhibitor. The second pharmacological moiety is an angiotensin converting enzyme (ACE) inhibitor. The two or more pharmacological moieties can be linked either by covalent bonds or by ionic interactions.

The invention also provides a method of reducing cardiovascular disease or cardiovascular disease-related conditions in an individual. The method involves administering to an individual with cardiovascular disease an effective amount of a compound, in which the compound has a first pharmacological moiety linked to, at least, a second pharmacological moiety and in which both pharmacological moieties, when active or when activated, act to reduce cardiovascular disease in the individual. The compounds of the invention can be delivered in a drug delivery device.

The use of the compounds of the invention is a convenience both for cardiovascular disease patients and for their physicians. Administration of the compounds of the invention also encourages improved patient compliance, which improves health.

The use of the compounds of the invention may also be a convenience for the pharmacist because use of the compounds permits simplified titration processes for drug preparation. Potentially, the cost of prepared compounds can be less than that of preparations of the individual components, after packaging costs are included.

Moreover, the compounds of the invention can reasonably be expected to potentiate the separate cardiovascular effects by additive or synergistic effect. Where such additive or synergistic effects occur, a reduction in adverse events can be achieved through lower dosage requirements of the separate moiety components. In general, an improved overall antihypertensive effect can be achieved where the ratio of the separate moiety components is superior to what is available in the absence of a fixed-dose combination

### BRIEF DESCRIPTION OF THE DRAWINGS

FIG. 1 is a diagram of the renin-angiotensin-aldosterone system.

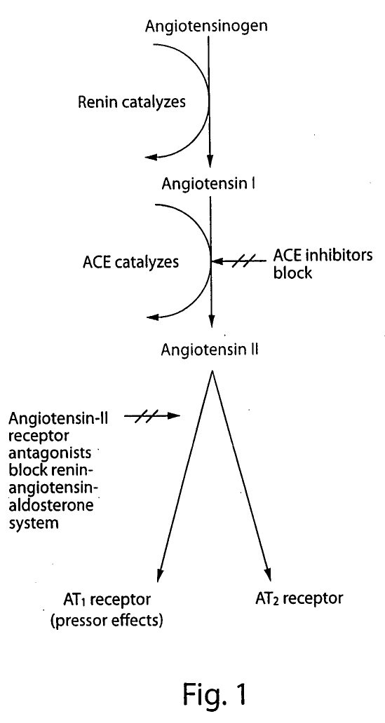

FIG. 2 is a diagram showing the similarity of structure among the HMGCoA reductase inhibitors (from Istvan, E. S. & Deisenhofer, J., Science 292: 1160-64 (2001)). The HMG-moiety is indicated by the dotted box, and the Kₘ value of HMG-CoA is indicated. Not shown in this figure are lovastatin (a type I HMGCoA reductase inhibitor) and pravastatin (a type II HMGCoA reductase inhibitor).

FIG. 3 is a diagram of a reaction scheme for fosinopril with simvastatin and fosinopril with lovastatin.

### DETAILED DESCRIPTION OF THE INVENTION

The invention provides a means of improving the pharmacology and delivery properties of pharmacologically active moieties, by conjugating them together to form a new compound. A “pharmacological moiety” is a compound that, when active or when activated, can cause an intended medical effect. Pharmacological moieties typically cause these effects when made to interact with a drug target (generally in the body of the individual to whom the compound is administered, particularly a human or mammal that is a model of a human disease or condition, but possibly also in an animal, such as a bird or mammal, in a veterinary administration of the compound). In this invention, the pharmacological moiety affects hypertension and hypertension-related diseases and conditions in animals, particularly mammals, more particularly, humans. Hypertension-related diseases are known in the medical arts and include damage to the blood vessels of the brain, heart, and kidneys, stroke, cardiac failure, renal failure and an increased the risk of myocardial infarct (MI).

The compound of the invention is a composition of at least two pharmacological moieties, either covalently linked to one another by a (usually labile) bond to form a single compound or ionically linked to one another to form a single working composition (see, U.S. Pat. No. 6,051,576, incorporated by reference).

A “prodrug” is a compound that is generally not pharmacologically active. However, when activated, typically in vivo by enzymatic or hydrolytic cleavage to convert the prodrug to a drug, the administration of the prodrug to the individual will have had the intended medical effect. Prodrugs are typically formed by chemical modification of a biologically active compound. One purpose of employing a prodrug, for oral administration, for example, is to increase intestinal or site-specific absorption. Another purpose is to reduce local side effects, such as gastrointestinal irritation. Prodrugs may also be used to increase transdermal absorption by enhancing permeation through topical membranes.

On this basis, prodrug formulations are not generally classified as sustained release dosage forms. However, the ability to bioreversibly modify the physicochemical properties of a drug (to create a prodrug compound) allows for better intestinal transport properties and hence can influence the drug blood levels versus time profile of the drug. Thus, prodrug formulations can be used as a strategy for sustained release and sustaining therapeutic levels of pharmacological moieties in an individual.

The compound of the invention contains a first and, at least, a second pharmacological moiety, and may also contain other pharmacological moieties (such as a third pharmacological moiety, and possibly a fourth pharmacological moiety, etc.). In one embodiment, the compound of the invention contains the first pharmacological moiety and the second pharmacological moiety in equimolar amounts. In a particular embodiment, the compound contains one first pharmacological moiety and one second pharmacological moiety.

The compound of the invention has several advantages for the treatment of hypertension. Among these are advantages for the patient, for the prescribing physician, and for the pharmacist (by reducing the number of active components in tablet formulation, each component having different properties). For the patient and the physician, the compound of the invention can enhance patient compliance by providing a convenient reduction in the number of pills to be taken. The compound of the invention can also be a drug compound that is superior to either pharmacological moiety, because the compound can have moieties with synergistic effects. The compound of the invention can also advantageously provide a pharmaceutical with improved bioavailability, since a single compound is administered (possibly a hydrophilic compound, for crossing the mucosa of an individual). Moreover, any patient population variance can be assessed by the physician in terms of a single compound, rather than two compounds. With the compound of the invention, differences in absorption between the pharmacological moieties do not lead to different doses.

Covalent Bonds Between Pharmacological Moieties. In one embodiment, the compounds of the invention are formed by covalent conjugation of two or more pharmacological moieties (Examples 1 and 2). Pharmacological moieties can be linked as a compound of the invention by reversible covalent bonds, such that at the desired site in the body, the covalently-linked pharmacological moieties are cleaved to regenerate the active forms of the pharmacological moieties, or the prodrug precursors to the drugs of interest. The rate of cleavage of the pharmacological moieties can be controlled by the type of the bond linking the pharmacological moieties, the choice of pharmacological moieties and the physical form of the compound.

The first and second pharmacological moieties may be covalently linked either by a direct covalent linkage or by an indirect covalent linkage, through a linker group (L group). This relationship can be generically expressed in the following Formula (I):
A₁-L-A₂ (I)
wherein A₁ and A₂ are the residues of the first pharmacological moiety and second pharmacological moiety, respectively, as defined above, and the linking (L) group is either a direct bond or a linker as described above. When the linking group is a direct bond, Formula (I above may be expressed more compactly as Formula (II):
A₁-A₂ (II)

When a compound of Formula I is exposed to physiologic fluids, such as blood plasma, it is subjected to hydrolysis.

Covalent bonds having an L group may be of (but are not limited to) the type: 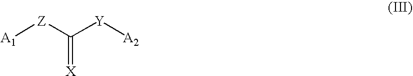
wherein Z is O, N, CH₂, CH₂O or CH₂S, Y is O, or N, and X is O or S. Covalent bonds can be, for example, ester, carbonate, cyclic phosphate ester or carbamate bonds. The physiologically labile linkage may be any linkage that is labile under conditions approximating those found in physiologic fluids, such as blood plasma. The linkage may be a direct bond (for instance, an amide, carbonate, carbamate, sulfonate, or a sulfamate linkage) or may be a linking group (for instance, a C₁-C₁₂ dialcohol, a C₁-C₁₂ hydroxylalkanoic acid, a C₁-C₁₂ hydroxyalkylamine, a C₁-C₁₂ diacid, a C₁-C₁₂ amino acid, or a C₁-C₁₂ diamine). The linkage may be a direct amide, carbonate, carbamate, and sulfamate linkages, and linkages via succinic acid, salicylic acid, diglycolic acid, and halides thereof. The linkages can be labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the compound has not been solubilized in a physiologic fluid. Thus, while some compounds of the invention may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g. non-aqueous solvents such as acetone). Thus, the labile linkages are such that, when the drug is dissolved in an aqueous solution, especially a physiologic fluid such as blood plasma, the hydrolysis reaction lies heavily on the side of the hydrolysis products.

Moreover, the covalent bond can be enzyme-specific, for example, enzymatically labile to esterases. Alternatively, the covalent bonds can be chemically labile (e.g., base catalyzed hydrolysis of the linkage).

The first pharmacological moiety or second pharmacological moiety, or both, can be moieties that either possess, or may be adapted to possess, a group that may be condensed with a linkage to form a hydrolytically labile bond. Examples of such groups are hydroxy (—OH) groups, amine (—NH₂ or —NH—) groups, acid (—COOH) groups, sulfonamide (—SO₂NH₂) groups, and sulfonate (—SO₃H) groups.

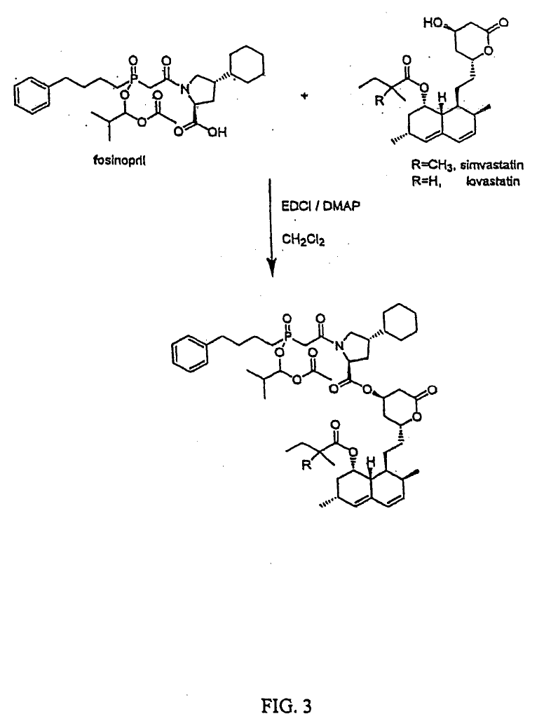

Exemplary reaction schemes for making the compounds of the invention are illustrated in FIG. 3 and Example 1. This scheme can be generalized by substituting other statins for simvastatin or lovastatin. Likewise, this scheme can be generalized by substituting other ACE inhibitors for fosinopril. This scheme can also be generalized by using appropriate linkers and agents as starting materials.

In general, where the first pharmacological moiety and the second pharmacological moiety are to be directly linked, the first pharmacological moiety is condensed with the second pharmacological moiety under conditions suitable for forming a linkage that is labile under physiologic conditions. In some cases, it is necessary to block some reactive groups on one, the other, or both of the moieties.

Where the moieties are to be covalently linked via a linker, such as succinic acid or diglycolic acid, the first pharmacological moiety can initially be condensed with the linker. Sometimes, it is advantageous to perform the reaction in a suitable solvent, such as acetonitrile, in the presence of suitable catalysts, such as carbodiimide and dimethylaminopyridine (DMAP), a nucleophilic catalyst, or under conditions suitable to drive off water of condensation or other reaction products (e.g. reflux), or a combination of two or more thereof. After the first moiety is condensed with the linker, the combined first moiety and linker may then be condensed with the second pharmacological moiety. Again, in some cases, it is advantageous to perform the reaction in a suitable solvent, such as acetonitrile, in the presence of suitable catalysts, or under conditions suitable to drive off water of condensation or other reaction products (e.g. reflux), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The active groups can be derivatized to increase their reactivity. For instance, where the first moiety is an acid and the second moiety is an alcohol (i.e. has a free hydroxyl group), the first moiety may be derivatized to form the corresponding acid halide, such as an acid chloride or an acid bromide. Other possibilities are known in the art for increasing yield, lowering production costs, improving purity, etc. of the compound of the invention by using conventionally derivatized starting materials to make compounds of the invention.

While diacids, dialcohols, amino acids, etc. are described above as being suitable linkers, other linkers are also within the scope invention. For example, while the hydrolysis product of a compound of the invention may comprise a diacid, the actual reagent used to make the linkage may be, for example, a diacetylhalide, such as a diacetylchloride or diacetylbromide, or a dianhydride. Other possible acid, alcohol, amino, sulfate, and sulfamoyl derivatives may be used as reagents to make the corresponding linkage.

In one advantageous embodiment of the invention, codrugs can be used to deliver the active metabolite to the person being treated. A prodrug may have no pharmacologic activity until metabolically converted into an active compound. When the metabolite of a drug produces the therapeutic effect, it is considered an “active metabolite”. For example, the first pharmacological moiety can be a hydroxy acid having structure similar to the product lactone hydrolysis of an HMGCoA reductase inhibitor, such as lovastatin, simvastatin, atorvastatin, or cerivastatin (but not pravastatin or fluvastatin). For the first pharmacological moiety, the open-ring hydroxy acid is often the active metabolite.

Ionic Bonds Between Pharmacological Moieties. In one embodiment, the compounds of the invention are formed by ionic interactions between two or more pharmacological moieties. See, U.S. Pat. No. 6,051,576, which is incorporated herein by reference.

Salt formation is an acid-base reaction involving either a proton-transfer or neutralization reaction and is therefore controlled by factors influencing such reactions. Theoretically, every compound that exhibits the appropriate acid or base characteristics can participate in salt formation. Particularly important is the relative strength of the acid or base and the acidity and basicity constants of the pharmacological moieties involved. These factors determine whether or not salt formation occurs and are a measure of the stability of the resulting salt. The salt form is known to influence a number of physico-chemical properties of the parent compound including dissolution rate, solubility, stability, and hygroscopicity. Salt formation is useful in pharmaceutical formulations since these properties, in turn, affect the availability and formulation characteristics of the drug.

To make the compound of the invention, the first pharmacological moiety is dissolved in an organic solvent together with an equivalent amount of the second pharmacological moiety. The solution is then evaporated under a nitrogen atmosphere at room temperature to a liquid/semi-solid viscous mass. The compound is then crystallized through the use of a suitable organic solvent such as alcohol, etc. The remainder of the liquid can be driven off through the continued application of heat. The compound is then formulated into any one of a number of known dosage forms or delivery systems by means known in the art. See, U.S. Pat. No. 5,385,941. See also, published PCT applications WO 99/11259 and WO 00/73298.

Moreover, the compound of the invention can be or can be formulated as a mineral acid salt, a carboxylic acid salt, or an amino acid salt.

First Pharmacological Moiety, HMGCoA reductase inhibitor. HMGCoA reductase inhibitors (also known as statins) are currently the most effective drugs in the battle against high cholesterol. The regulation cholesterol biosynthesis has long been the subject of intensive research because of its connection with atherosclerosis, cerebrovascular and coronary heart disease. Control of cholesterol synthesis occurs mainly at the first committed step in the pathway, catalyzed by 3-hydroxy-3-methylglutaryl CoA (HMGCoA) reductase.

Statins block hydroxymethylglutaryl-CoA reductase (EC 1.1.1.34), an enzyme needed in the formation of cholesterol. Other names for the enzyme include hydroxymethylglutaryl coenzyme A reductase (reduced nicotinamide adenine dinucleotide phosphate); 3-hydroxy-3-methylglutaryl-CoA reductase; β-hydroxy-β-methylglutaryl coenzyme A reductase; hydroxymethylglutaryl CoA reductase (NADPH); S-3-hydroxy-3-methylglutaryl-CoA reductase; NADPH-hydroxymethylglutaryl-CoA reductase; HMGCoA reductase-mevalonate:NADP-oxidoreductase (acetylating-CoA); 3-hydroxy-3-methylglutaryl CoA reductase (NADPH) and (R)-mevalonate:NADP oxidoreductase (CoA-acylating). The enzyme catalyzes the conversion of (s)-3-hydroxy-3-methylglutaryl-CoA+2 NADPH₂ to (R)-mevalonate+CoA+2 NADP.

Among the statin class of drugs are Lipitor® (atorvastatin); Pravachol® (pravastatin); Zocor® (simvastatin); Mevacor® (lovastatin); Lescol® (fluvastatin); and Baycol® (Cerivastatin) and Crestor®, formerly ZD4522 (rosuvastatin).

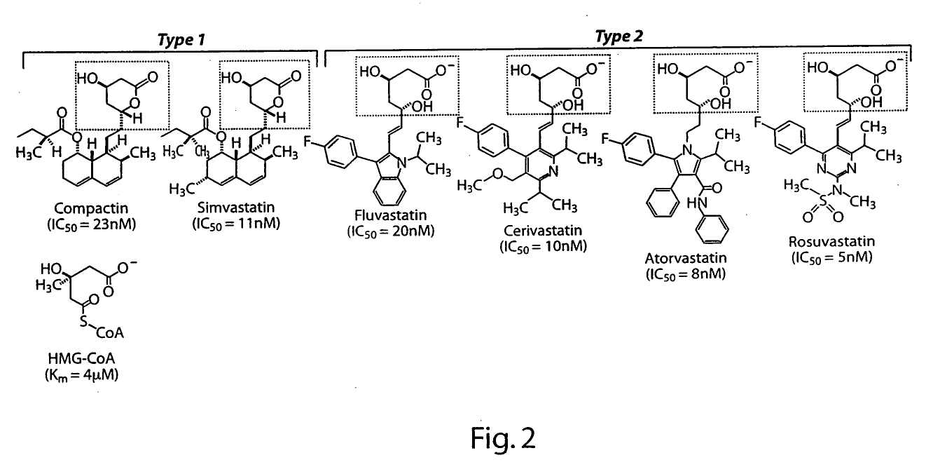

The structure of the statin class of compounds is known to those of skill in the pharmacological arts. All statins share an HMG-like moiety as shown in FIG. 2. The statins share rigid, hydrophobic groups that are covalently linked to the HMGCoA-like moiety. Lovastatin, pravastatin, and simvastatin resemble the substituted decalin-ring structure of compactin (also known as mevastatin). Istvan, E. S. & Deisenhofer, J., Science 292: 1160-64 (2001) classify this group of inhibitors as type 1 statins. Fluvastatin, cerivastatin, atorvastatin, and rosuvastatin are fully synthetic HMGCoA reductase inhibitors with larger groups linked to the HMG-like moiety. Istvan & Deisenhofer refer to these inhibitors as type 2 statins. The additional groups range in character from very hydrophobic (e.g., cerivastatin) to partly hydrophobic (e.g., rosuvastatin). All statins are competitive inhibitors of HMGR with respect to binding of the substrate HMG-CoA, but not with respect to binding of NADPH. The Kᵢ (inhibition constant) values for the statin-enzyme complexes range between 0.1 to 2.3 nM, whereas the Michaelis constant, Kₘ, for HMG-CoA is 4 μM.

Istvan & Deisenhofer have determined how the structures of the catalytic portion of human HMGCoA reductase are complexed with different statins. The bulky, hydrophobic compounds of statins occupy the HMG-binding pocket and part of the binding surface for CoA. Thus, access of the substrate HMG-CoA to HMG-CoA reductase is blocked when statins are bound.

Statins have proven to be very effective at lowering blood cholesterol levels and also at preventing heart attacks, which is one of the main consequences of high cholesterol levels. The process by which cholesterol causes the damage is known as atherosclerosis and involves the build-up of cholesterol-containing plaques in the walls of the arteries, which can eventually block them altogether. The plaque in the arteries supplying the heart results in a heart attack, and in the arteries supplying the brain, causes stroke.

Statins generally have few side effects, and help not only to lower overall cholesterol, LDL (so-called “bad”) cholesterol and triglycerides, but also to increase HDL (so-called “good”) cholesterol. Primary and secondary prevention trials have shown that use of statins to lower an elevated low-density lipoprotein cholesterol level can substantially reduce coronary events and death from coronary heart disease. Strong evidence in support of lipid lowering as a means of secondary coronary heart disease prevention comes from three large trials, the Scandinavian Simvastatin Survival Study (4S study) (Lancet 344:1383-9 (1994)), Long-Term Intervention with Pravastatin in Ischaemic Disease (LIPID) study (Sacks F M, et al., N. Engl. J. Med. 335:1001-9 (1996)) and the Cholesterol and Recurrent Events (CARE) trial (N. Engl. J. Med. 339:1349-57 (1998)) in which treatment with HMG-CoA reductase inhibitors (statins) reduced coronary events and reduced mortality. Studies have also shown that some statins are effective in preventing not only recurrent heart attacks, but first heart attacks as well. Some statins are also effective in reducing the risk of strokes. New studies have shown that even people with ordinary cholesterol levels might benefit from taking cholesterol-lowering drugs. Statin therapy is indicated for primary prevention in hypertensive subjects up to 70 years old with a cholesterol level of greater than 5 mmol/L and a 10-year coronary artery disease risk of greater than 30%. Ramsay, L. E., J. Human Hypertension 13: 569-592 (1999) and Ramsay, L. E., British Med. J. 319: 630-635 (1999).

Unfortunately, statin use is under-prescribed. The National Cholesterol Education Program has promulgated guidelines for cholesterol screening and treatment (Arch. Intern. Med. 148:36-69 (1988) and National Cholesterol Education Program, NIH publication no. 97-3794 (1997)). Thus far, however, primary care physicians have inadequately adopted these guidelines in clinical practice (see, Am. Fam. Physician 63: 309-20, 323-4 (2001)). Moreover, even when prescribed, patient compliance is a problem.

Some typical daily dosages for oral administration of statins are shown in the Table 1 below:

TABLE 1

| Statin | Usual Daily Dose Range | |
|---|---|---|
| Lovastatin | 10-80 | mg/day |
| Simvastatin | 5-40 | mg/day |
| Pravastatin | 10-80 | mg/day |
| Fluvastatin | 20-40 | mg/day |

Second Pharmacological Moiety; Angiotensin Converting Enzyme (ACE) Inhibitor. Angiotensin-converting enzyme (ACE) inhibitors block angiotensin-converting enzyme (ACE), which is necessary to produce a substance that causes blood vessels to tighten. As a result, they relax blood vessels. This lowers blood pressure and increases the supply of blood and oxygen to the heart. ACE inhibitors help control tissue damage caused by activation of the renin-angiotensin-aldosterone system (RAAS) following cardiac injury.

Overexpression of renin and its metabolic products predisposes to increased blood pressure and even frank hypertension, as well as target organ damage. Renin reacts with angiotensinogen to produce the decapeptide angiotensin I, which is biologically inactive. Angiotensin I is cleaved by a variety of enzymes, including angiotensin converting enzyme (ACE) to generate angiotensin II, an octapeptide that is responsible for most of the known biological activity of the system.

Angiotensin II elevates blood pressure by a variety of mechanisms, including direct vasoconstriction, potentiation of sympathetic nervous system activity of both central and peripheral levels, stimulation of aldosterone synthesis and release with consequent sodium and fluid retention by the kidney and stimulation of arginine vasopressin release. In addition, angiotensin II has a variety of actions that damage blood vessels directly. Angiotensin II also plays a role in the vascular injury response, stimulating leukocyte adhesion to the site of injury and favoring superoxide and peroxynitrite formation and proliferation and migration of various cell types toward the luminal site of injury, which eventually causes cellular components of the arterial wall to transform their phenotypes, resulting in atherosclerotic plaque or fibrous neointima formation.

Angiotensin converting-enzyme inhibitors are active-site directed inhibitors. ACE inhibitors are 2-methylproprionyl-L-proline analogues that exert their effect by forming a zinc ligand. Piepho, R W., Am. J. Health Syst. Pharm. 57 Suppl 1:S3-7 (2000). They utilize all the critical binding interactions of the substrate and convert the catalytic interaction with the zinc atom into an effective binding interaction. Ondetti, M. S., Circulation 77(6 Pt 2):174-8 (1988). Three subclasses of angiotensin converting-enzyme inhibitors are the sulfhydryl-containing inhibitors such as captopril and its analogs and prodrugs, carboxyalkyldipeptides such as enalapril and its analogs, and phosphorous-containing inhibitors such as fosinopril. The functional group binding to angiotensin converting enzyme through the zinc moiety is the primary structural difference among this class of pharmacological agents. Also, the sulfhydryl-containing inhibitors such as captopril undergo a metabolic process to interact with endogenous sulfhydryl-containing compounds like glutathione and proteins, to form reversible disulfides, which can serve as depot forms of the drug.

There are many ACE inhibitors available, including benazepril (Lotensin®), captopril (Capoten®), enalapril (Vasotec®), fosinopril (Monopril®), lisinopril (Prinivil®, Zestril®), quinapril (Accupril®), ramipril (Altace®), and trandolapril (Mavik). Some other ACE inhibitors include cilazapril, enalaprilat, moexipril, and perindopril. Some other commonly used brand names are, in the U.S., Aceon®, Prinivil®, Univasc®, and Zestril®. Other common brand names are, in Canada, Coversyl and Inhibace.

In addition, a class of “super” ACE inhibitors is the vasopeptidase inhibitors, such as omapatrilat. Like ACE inhibitors, vasopeptidase inhibitors block angiotensin, but they also neutralizes neutral endopeptidase, causing blood vessels to relax. A typical dose is 40 mg of omapatrilat daily.

ACE inhibitors belong to the class of drugs called antihypertensives. Administration of ACE inhibitors is known to improve symptomatic status in all grades of heart failure. Brown N J & Vaughn, D E. Circulation (97:1411-1420 (1998). ACE inhibitors also reduce mortality in both heart failure and the postmyocardial infarction period, as well as during the slow progression to end-stage renal disease. Many ACE inhibitors have undergone the appropriate clinical trials carry an indication for their use in heart failure.

Benazepril, captopril, cilazapril, enalapril, enalaprilat, fosinopril, lisinopril, moexipril, perindopril, quinapril, rampril, and trandolapril are antihypertensives. Benazepril, captopril, cilazapril, enalapril, fosinopril, lisinopril, quinapril, ramipril, and trandolapril are used as valodilators, or for congestive heart failure. Lisinopril, captopril, ramipril, and trandolapril are used in some patients after a heart attack. After a heart attack, some of the heart muscle is damaged and weakened. The heart muscle may continue to weaken as time goes by. This makes it more difficult for the heart to pump blood. Captopril, ramipril, and trandolapril help slow down the further weakening of the heart. Captopril is also used to treat kidney problems in some diabetic patients who use insulin to control their diabetes.

The ACE inhibitors captopril and lisinopril are not prodrugs. Other ACE inhibitors require activation through hepatic biotransformation.

Some typical daily dosages for administration of angiotensin converting enzyme inhibitors are shown in Table 2 below:

TABLE 2
ACE
INBITOR

ADULT ORAL DOSE FOR THE TREATMENT OF HYPERTENSION

| | |
|---|---|
| benazepril | 10 to 40 mg a day, taken as a single dose or divided |
| | into two doses |
| captopril | 25 to 50 mg, two to three times a day |
| cilazapril | 2.5 to 10 mg, once a day |
| enalapril | 5 mg to 40 mg a day, taken as a single dose or divided |
| | into two doses |
| fosinopril | 10 to 40 mg, once a day |
| lisinopril | 10 to 40 mg, once a day |
| moexipril | 7.5 mg to 30 mg a day, taken as a single dose or |
| | divided into two doses |
| perindopril | 4 mg to 16 mg a day, taken as a single dose or divided |
| | into two doses |
| quinapril | 10 to 80 mg a day, taken as a single dose or divided |
| | into two doses |
| ramipril | 2.5 to 20 mg a day, taken as a single dose or divided |
| | into two doses |
| trandolapril | 1 to 4 mg a day, taken as a single or divided into |
| | two doses |

Adult injection dosage for treatment of hypertension

| | |
|---|---|
| enalapril | 1.25 mg every six hours, injected into a vein |

Diagnosis of Hypertension. Diagnosis of hypertension and hypertension-related conditions, and the identification of individuals who would benefit by medical treatment for hypertension, are standard medical diagnoses. Further guidance may be obtained from The International Society of Hypertension and the World Health Organization (J. Hypertension 17: 151-183 (1999)), which suggest that young, middle-aged or diabetic subjects should be treated to a target blood pressure less than 130/80 mm Hg and the elderly to less than 140/90 mm Hg. The British Hypertension Society guidelines recommend the initiation of treatment with a systolic blood pressure greater than or equal to 160 mm Hg or a diastolic blood pressure greater than or equal to 100 mm Hg. The British Hypertension Society suggests that subjects with a blood pressure between 140-159 mm Hg systolic and 90-99 mm Hg diastolic should be treated in the presence of other risk factors, aiming for a target blood pressure less than 140/85 mm Hg. In diabetic patients the British Hypertension Society aim is to reduce blood pressure to less than 140/80 mm Hg. Other guidance is provided in Table 3.

TABLE 3
Diagnostic Classification for Hypertension by JNC-V (1993)

| Category | Systolic(mm HG) | Diastolic(mm Hg) |
|---|---|---|
| Normal | <130 | <85 |
| High normal | 130-139 | 85-89 |

Hypertension

| | | |
|---|---|---|
| Stage 1 | 140-159 | 90-99 |
| Stage 2 | 160-179 | 100-109 |
| Stage 3 | 180-209 | 110-119 |
| Stage 4 | >210 | >120 |

Dosages and Formulations for Oral Administration. Dosages for administration of the compounds of the invention may be calculated by those of skill in the art (see, Goodman & Gilman, The Pharmacological Basis of Therapeutics, 8th Ed. (Pergamon Press, NY, 1990); and The Merck Index, 11th Ed. (Merck and Co., Inc., Rahway, N.J. 1989); both incorporated herein by reference). Dosages are preferably in the range of about 1 to about 500 mg/kg body weight, and are administered preferably 1 to 2 times a day. Additional guidance for the appropriate dosage for oral administration of compounds may be found in the dosages for the first pharmacological moiety and second pharmacological moiety, respectively as shown in Tables 1 and 2, respectively. From published studies of administration of the first pharmacological moiety and second pharmacological moiety and the information known to those of skill in the art, appropriate therapeutic ranges for administration of the compounds of the invention can reasonably be estimated. As one example, from the information provided in Tables 1 and 2, the compound of the invention can be administered with a range of effective dosages. The lower end of the range can be 1 μg/day, more particularly 1 mg/day, more particularly 2.5 mg/day, more particularly 4 mg/day, more particularly 5 mg/day, more particularly 7.5 mg/day, more particularly 10 mg/day, or 25 mg/day. The upper end of the range can be 150 mg/day, more particularly 100 mg/day, more particularly 80 mg/day, more particularly 40 mg/day, more particularly 20 mg/day, more particularly 16 mg/day, or 4 mg/day. The compound of the invention is administered only once or at most twice a day.

The compounds of the invention are labile when dissolved in bodily fluids and are rapidly hydrolyzed to regenerate the two active parent drugs. In the solid form however, they are stable, even in an aqueous environment because in order to hydrolyze they must first be in solution.

Other Dosages and Formulations. The method of the invention advantageously employs a compound of the invention, which may be delivered to an individual in need thereof in an art recognized manner, such as via intravenous, subcutaneous, intramuscular or other parenteral mode of injection, or by surgical implantation. Although intravenous injection is possible, the properties of the compounds of the invention make them well-suited for subcutaneous or intramuscular implantation or injection into soft tissue.

The compounds of the invention can also be formulated as suspensions (nanoparticle size range) and upper size limitations are only imposed the application method under consideration.

In an embodiment of the invention, a compound of the invention is prepared in a solid form, such as a pellet that may be directly injected. Pellets of a compound of the invention can slowly release drugs in solution or into bodily fluids, reflecting the low solubility of the conjugated forms. Pellets may be formulated from the compounds alone or with implantable, bioerodible substances such as polylacetic acid and polyglycolic compounds. Pellets may be formulated by methods known in the art and may contain 0.1 to about 100% of the composition.

In other embodiments of the invention, the compound of the invention is prepared in an anhydrous solution or suspension, for instance in vegetable oil, such as palm oil, and injected intramuscularly. The compound of the invention may be administered in injectable form such as in liposomes, liquids, suspensions, microspheres or nanoparticles. Preparation of such aqueous solutions, liposomes, emulsion and suspensions are known to those of ordinary skill in the art (see, Remington's Pharmaceutical Sciences, 18th Ed. (Mack Publishing Co., Easton, Pa., 1990)).

In another embodiment, the compound is an oral formulation, such as in capsules, tablets, or gelcaps. In yet another embodiments of the invention, the compound is in a topically applicable form, such as a transdermal patch, ointment, cream, suspension, liquid, elixir or eye drop (see, Remington's Pharmaceutical Sciences, 18th Ed. (Mack Publishing Co., Easton, Pa., 1990)).

Controlled Delivery Systems. In one embodiment of the invention, compounds of the invention (either solid, liquid or colloidal) are contained in controlled delivery systems for a controlled or sustained release of compounds of the invention for a systemic or local pharmacological or physiological effect relating to hypertension and hypertension-related disease states. Such disease states are known to those of ordinary skill in the art (see, Goodman & Gilman, The Pharmacological Basis of Therapeutics, 8th Ed. (Pergamon Press, NY, 1990); and The Merck Index, 11th Ed. (Merck and Co., Inc., Rahway, N.J. 1989); both incorporated herein by reference).

The controlled delivery system is preferably chosen such that the compound of the invention has a rate of diffusion from the polymer matrix under physiologic conditions be not rate-limited by the permeability of the polymer matrix. See, U.S. Pat. No. 6,051,576, incorporated by reference, for a discussion of controlled delivery systems.

Formulations of the compounds of the invention may also contain several other substituents to optimize release, bioavailability or appearance and may be used in sustained release devices or systems. Such substituents are known to those of ordinary skill in the art and, for example, are set forth in Remington's Pharmaceutical Sciences, 18th Ed. (Mack Publishing Co., Easton, Pa., 1990). Furthermore, the compounds may be conjugated to another agent to reduce the undesirable effects such as isoniazid with pyroxidine. Another embodiment of the invention is a compound of the invention formulated with other drug or prodrug molecules.

A compound of the invention may also be formulated in bioerodible or nonbioerodible delivery systems to further control their release. Such bioerodible systems may include polylacetic acid (bioerodible) to form a film around, or a matrix with a compound of the invention to further improve the pharmaceutical properties. Polylacetic acid can be formulated in solutions of 2, 5, and 10% polylacetic acid, and has been used to produce pellets attached to sutures. A totally bioerodible sustained release system for pharmacologically active agents can be composed of a compound of the invention in a formulation with another bioerodible substance such as polyvinyl acid, polyanyhydride, collagen, or polyalkylcyanoacrylates such as polybutylcyanoacrylate. 2% polyvinyl alcohol has been used to coat pellets of for subconjunctival delivery. Polybutyl cyanoacrylate (bioerodible) has also been used to form a matrix with pellets.

In another embodiment of the invention, compounds of the invention are contained in a nonerodible matrix or reservoir system containing natural or synthetic polymers that are biologically compatible with and essentially insoluble in body fluids. Such materials include for example, but are not limited to polyvinyl acetate, polyvinyl alcohol, cross-linked polyvinyl butyrate, ethylene ethyl acrylate copolymer, polyethyl hexyl acrylate, polyvinyl chloride, polyvinyl acetals, plasticized ethylene vinyl acetate copolymer, ethylene vinyl chloride copolymer, polyvinyl esters, polyvinyl butyrate, polyvinyl formal, polyamides, polymethyl methacrylate, polybutyl methacrylate, plasticized polyvinyl chloride, plasticized nylon, plasticized soft nylon, plasticized polyethylene terethphalate, natural rubber, polyisoprene, polyisobutylene, polybutadiene, polyethylene, polytetrafluoroethylene, polyvinylidine, chloride, polyacrylonitrile, cross-linked polyvinyl pyrrolidone, polytrifluorochloroethylene, chlorinated polyethylene, poly(1,4-isopropylidne diphenylene carbonate), vinylidine chloride, acrylonitrile copolymer, vinyl chloride-diethyl fumarate copolymer, silicone rubbers (especially medical grade polydimethylsiloxanes, ethylene-propylene rubber, silicone-carbonate copolymers, vinylidine chloride-vinyl chloride copolymer, vinyl chloride-acrylonitrile copolymer and vinylidine chloride acrylonitrile copolymer.

Systems containing the compounds of the invention may be directly implanted in a site in the vicinity of the surgical incision, in the vicinity of soft tissues, or both. In some embodiments of the invention, it may be desirable to combine a compound of the invention with one or more polymer vehicle. Such polymer vehicle may be any physiologically tolerated polymer, such as a bioerodible or a non-bioerodible polymer.

A polymer useful in a composition of the invention includes any biologically tolerated polymer that is permeable to a compound of the invention or that is bioerodible so that it releases the compound of the invention in a sustained-release manner. In preferred embodiments of the invention, the polymer has a permeability such that the permeability is not the principal rate determining factor in the rate of release of the compound of the invention from the polymer. In some embodiments of the invention, the polymer is non-bioerodible. Examples of nonbioerodible polymers useful in the invention include polyvinyl alcohol and polyurethane. In other embodiments of the invention, the polymer is bioerodible. Examples of bioerodible polymers useful in the invention include polyanhydride, polylacetic acid, polyglycolic acid, polyorthoester, polyalkylcyanoacrylate or derivatives and copolymers thereof. Those of skill in the art will recognize that the choice of bioerodibility or nonbioerodibility of the polymer depends upon the final physical form of the system, as described in greater detail below. Other exemplary polymers include polysilicone and polymers derived from hyaluronic acid. The skilled artisan will understand that the polymer is prepared under conditions suitable to impart permeability such that it is not the principal rate determining factor in the release of the low solubility agent from the polymer.

Moreover, suitable polymers include naturally occurring materials (such as collagen or hyaluronic acid) or synthetic materials that are biologically compatible with bodily fluids and mammalian tissues, and essentially insoluble in bodily fluids with which the polymer will come in contact. In addition, the suitable polymers essentially prevent interaction between the low solubility agent dispersed/suspended in the polymer and proteinaceous components in the bodily fluid. The use of rapidly dissolving polymers or polymers highly soluble in bodily fluid or which permit interaction between the low solubility agent and proteinaceous components are to be avoided since dissolution of the polymer or interaction with proteinaceous components would affect the constancy of drug release.

Other suitable polymers include polypropylene, polyester, polyethylene vinyl acetate (PVA), polyethylene oxide (PEO), polypropylene oxide, polycarboxylic acids, polyalkylacrylates, cellulose ethers, polyalkyl-alkyacrylate copolymers, polyester-polyurethane block copolymers, polyether-polyurethane block copolymers, polydioxanone, poly-(β-hydroxybutyrate), polylacetic acid (PLA), polycaprolactone, polyglycolic acid, and PEO-PLA copolymers.

Further suitable polymers are set forth in U.S. Pat. No. 6,051,576, incorporated herein by reference.

The details of one or more embodiments of the invention are set forth in the accompanying description above. Although any methods and materials similar or equivalent to those described herein can be used in the practice or testing of the invention, the preferred methods and materials are now described. Other features and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The following examples are presented in order to more fully illustrate the preferred embodiments of the invention. These examples should in no way be construed as limiting the scope of the invention, as defined by the appended claims.

### EXAMPLE 1

Fosinopril with Simvastin or Lovastatin (FIG. 3)

Codrug of fosinopril with simvastatin. Fosinopril (74 mg), EDCI (24 mg) and catalytic amount of DMAP were dissolved in 2 ml of anhydrous dichloromethane at 0-5° C. under argon. After 15 min., simvastatin (44 mg) was added and the resulting solution was stirred in an ice bath for 15 min. and then at room temperature overnight. The reaction mixture was diluted with dichloromethane and washed subsequently with sodium bicarbonate aq., water, brine and dried over anhydrous sodium sulfate. Evaporation of the solvent afforded colorless oil, which was purified by preparative TLC to yield 40 mg of the codrug.

¹H-NMR (CDCl₃), 0.88 (t, 3H), 1.20 (m, 9H), 1.22 (m, 6H), 3.30-3.60 (bm, 1H), 4.48 (m, 1H), 4.57 (m, 1H), 5.32 (m, 1H), 5.42 (m, 1H), 5.58 (m, 1H), 5.84 (m, 1H), 6.05 (d, 1H), 6.40 (dd, 1H), 7.22 (m, 5H).

Codrug of fosinopril with lovastatin. To a stirred solution of fosinopril (320 mg) in 7 mL of anhydrous dichloromethane at 0-2° C. was added EDCI (133 mg) and DMAP (5 mg). After 4 minutes lovastatin (135 mg) was added and the resulting mixture was stirred in an ice-bath for 5 hr and left in refrigerator overnight. The solvent was evaporated to dryness and the residue was dissolved in ethyl acetate. The organic solution washed with water, brine and dried over anhydrous sodium sulfate. Evaporation afforded 413 mg of the colorless crude product, which was purified by passing through short pad of silica gel to yield 300 mg of codrug.

¹H-NMR (CDCl₃), 3.29 (m, 1H), 3.50 (m, 1H), 3.87 (t, 1H), 4.47 (m, 2H), 5.24 (m, 1H), 5.38 (m, 1H), 5.53 (m, 1H), 5.78 (m, 1H), 5.99 (m, 1H), 6.32 (dd, 1H), 7.16 (m, 2H), 7.26 (m, 3H).

The foregoing description has been presented only for the purposes of illustration and is not intended to limit the invention to the precise form disclosed, but by the claims appended hereto.

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

We claim:

1. A compound comprising a first pharmacological moiety covalently linked to a second pharmacological moiety through a physiologically labile linkage, or a salt thereof,
(a) wherein the first pharmacological moiety is an HMGCoA reductase inhibitor or a prodrug of an HMGCoA reductase inhibitor; and
(b) wherein the second pharmacological moiety is an angiotensin converting enzyme (ACE) inhibitor or a prodrug of an angiotensin converting enzyme inhibitor.

2. The compound of claim 1, wherein the compound, when exposed to physiologic fluids, decomposes to form an HMGCoA reductase inhibitor and an angiotensin converting enzyme inhibitor.

3. The compound of claim 1, wherein the first pharmacological moiety is selected from atorvastatin, pravastatin, simvastatin, lovastatin, fluvastatin, cerivastatin, and rosuvastatin.

4. The compound of claim 1, wherein the second pharmacological moiety is selected from telmisartan, losartan, valsartan, irbesartan, candesartan cilexetil and other angiotensin converting enzyme inhibitors.

5. The compound of claim 1, wherein the compound contains the first pharmacological moiety and the second pharmacological moiety in equimolar amounts.

6. The compound of claim 1, wherein the first pharmacological moiety is covalently linked to the second pharmacological moiety through one or more physiologically labile covalent bonds selected from amide, carbonate, carbamate, ether, ester, sulfonate, and sulfamate bonds.

7. The compound of claim 1, wherein the compound is a mineral acid salt, a carboxylic acid salt, or an amino acid salt.

8. The compound of claim 1, wherein an active drug is regenerated upon cleavage of a covalent bond between the first pharmacological moiety and the second pharmacological moiety.

9. The compound of claim 1, wherein a prodrug is produced upon cleavage of a covalent bond between the first pharmacological moiety and the second pharmacological moiety.

10. The compound of claim 1, wherein an active metabolite is produced upon cleavage of a covalent bond between the first pharmacological moiety and the second pharmacological moiety.

11. The compound of claim 1, in an injectable form.

12. The compound of claim 11, wherein the injectable form is selected from liposomes, suspensions, microspheres and nanoparticles.

13. The compound of claim 1, in a solid form.

14. The compound of claim 1, in a systemic form.

15. The compound of claim 14, wherein the systemic form is selected from capsules, tablets, and gelcaps.

16. The compound of claim 1, in a topically applicable form.

17. The compound of claim 16, wherein the topically applicable form is selected from a transdermal patch, ointment, cream, suspension, liquid, elixir and eye drop.

18. The compound of claim 1, wherein the compound is fixed to an implantable device.

19. The compound of claim 1, wherein the compound is coated on an implantable device.

20. The compound of claim 1, further comprising an erodible delivery vehicle.

21. The compound of claim 1, further comprising a nonerodible delivery vehicle.

22. A method of treating cardiovascular disease, comprising:
administering to an individual having cardiovascular disease a pharmaceutically effective amount of compound comprising a comprising a first pharmacological moiety covalently linked to a second pharmacological moiety through a physiologically labile linkage, or a salt thereof,
(a) wherein the first pharmacological moiety is an HMGCoA reductase inhibitor or a prodrug of an HMGCoA reductase inhibitor; and
(b) wherein the second pharmacological moiety is an angiotensin converting enzyme inhibitor or a prodrug of an angiotensin converting enzyme inhibitor.

23. The method of claim 22, wherein the individual is a mammal.

24. The method of claim 22, wherein the individual is a human.

25. The method of claim 22, wherein the compound is administered by a method selected from injection, inhalation, implantation, applied as a nasal spray, applied rectally, applied vaginally, ingested orally and applied topically.

26. An article of manufacture, comprising:
(a) a compound comprising a comprising a first pharmacological moiety covalently linked to a second pharmacological moiety through a physiologically labile linkage, or a salt thereof,
(i) wherein the first pharmacological moiety is an HMGCoA reductase inhibitor or a prodrug of an HMGCoA reductase inhibitor; and
(ii) wherein the second pharmacological moiety is an angiotensin converting enzyme inhibitor or a prodrug of an angiotensin converting enzyme inhibitor; and
(b) a polymer matrix, wherein the compound is in the polymer matrix.

27. The article of manufacture of claim 26, wherein the compound has a rate of diffusion from the polymer matrix under physiologic conditions that is not rate-limited by the permeability of the polymer matrix.

28. The article of manufacture of claim 26, wherein the compound, when exposed to physiologic pH, decomposes to form a first compound corresponding to said first pharmacological moiety, and a second compound corresponding to the second pharmacological moiety.

29. The article of manufacture of claim 26, wherein the polymer is a bioerodible polymer.

30. The article of manufacture of claim 26, wherein the polymer is a non-bioerodible polymer.
